(19) United States
(12) Patent Application Publication
(10) Pub. No.: US 2016/0184439 A1
(43) Pub. Date: Jun. 30, 2016

(54) ORALLY DISINTEGRATING FILM PREPARATION CONTAINING DONEPEZIL OR PHARMACEUTICALLY ACCEPTABLE SALT THEREOF, AND PREPARATION METHOD THEREFOR

(71) Applicant: SEOUL PHARMA. CO., LTD., Gyeonggi-do (KR)

(72) Inventors: Hyun Soo KIM, Gyeonggi-do (KR); Won Young JANG, Gyeonggi-do (KR); Jin Hee UM, Gyeonggi-do (KR); Sang Pil SIM, Gyeonggi-do (KR); Kyu Jeong YEON, Seoul (KR); Jin Gyu PARK, Gyeonggi-do (KR)

(21) Appl. No.: 14/906,868

(22) PCT Filed: Aug. 1, 2014
(86) PCT No.: PCT/KR2014/007124

§ 371 Date: Jan. 21, 2016

(30) Foreign Application Priority Data
Aug. 2, 2013 (KR) 10-2013-0091951

(51) Int. Cl. A61K 47/40; A61K 9/00; A61K 47/02; A61K 31/445; A61K 47/36

(52) U.S. Cl. CPC A61K 47/40; A61K 31/445; A61K 47/36; A61K 47/02; A61K 9/0056

(57) ABSTRACT

The present invention relates to an oral dissolving film formulation containing donepezil and a manufacturing method thereof and, more specifically, an oral dissolving film formulation for treating dementia including donepezil or acid addition salt thereof, cyclodextrin or derivatives thereof, alginic acid or salts thereof and a substrate for forming films, which is manufactured by adding a substrate for forming films to a liquid solution manufactured by firstly adding donepezil or acid addition salt thereof and secondly adding alginic acid or salts thereof to a solution in which cyclodextrin or derivatives thereof is melted, covering bitter taste and paralysis of donepezil.

### FIELD

The present invention relates to an oral dissolving film formulation containing donepezil or pharmaceutically acceptable salts thereof and a preparation method for the same. More preferably, it relates to an oral dissolving film containing donepezil or pharmaceutically acceptable salts thereof, which is the oral dissolving film in which paralysis and bitter taste are removed, and in particular, it relates to the oral dissolving film having a high quality in which a production efficiency is high and an impurity is reduced.

### BACKGROUND

Donepezil being an effective ingredient of the present invention is a compound represented by the below Chemical Formula, and donepezil or pharmaceutically acceptable salts thereof has been known to act as a cholinergic agent and be a treating agent for dementia in the form of Alzheimer:

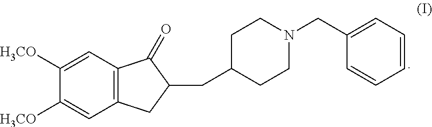 [SMILES: COc1cc2c(cc1OC)C(=O)C(CC1CCN(Cc3ccccc3)CC1)C2]

A preparation method for the same is disclosed in European Patent No. 296560 (1988) and U.S. Pat. No. 4,895,841 (1990), etc. and, it is known in Korean Patent Nos. 522574, 953038, 1062973, and the like.

As the salts thereof, oxalate salt is described in Korean Laid-open Patent No. 10-2007-0116996 and maleate salt is described in Korean Laid-open Patent No. 10-2007-0083679.

But, since donepezil or pharmaceutically acceptable salts thereof has a characteristic being administered to a patient with dementia, when it is prepared into a general oral dose preparation, for example, a general tablet or capsule, a drug compliance thereof can be declined due to a resistance at the time taking a dose or a difficulty in swallowing. Therefore, there have been performed approaches to make it to a rapid dissolving formulation, for example, Orally Disintegrating Table (ODT) or Orally Disintegration Film (ODF), but due to paralysis and bitter taste formulations developed until now have many rooms for development, there remains a need for new formulation which comprises donepezil or pharmaceutically acceptable salts thereof, while does not have any bitter taste and paralysis.

As reviewed in the above, it is important to mask bitter taste and paralysis in the prior art to which the present subjects, and as the relevant Patent techniques, roughly, the following techniques are shown.

Korean Registration Patent Publication No. 693266 describes the method for reducing a binding rate in a bitter taste receptor of tongue by reducing a free form of donepezil in saliva due to an interaction of donepezil hydrochloride with carrageenan, chondroitin sulfate, and dextran sulfate, and a granule, powder, syrup, and the like containing the same, but, since it is difficult to completely remove free form of donepezil, it was difficult to sufficiently mask the unpleasant taste and property such as bitter taste, paralysis, etc. of donepezil hydrochloride.

Korean Registration Patent Publication No. 801236 describes the method for masking the unpleasant taste of donepezil hydrochloride by adding polyvinylpyrrolidone, etc. to donepezil hydrochloride, but has a disadvantage that its formulation is limited to a liquid.

In addition, Korean Registration Patent Publication No. 1124796 describes a method for obtaining donepezil resin complex by adsorbing donepezil to a cation exchange resin, methacrylic divinylbenzene resin, and an oral dissolving formulation comprising the donepezil resin complex, but it is also insufficient to achieve a perfect masking of bitter taste.

Further, Korean Laid-open Patent Publication No. 10-2009-0080037 describes a persistent mucous membrane non-adhesive film formulation comprising donepezil hydrochloride and hydrophilic binder, aqueous diluent cyclodextrin or derivatives thereof and having Tₘₐₓ of 3-4 hours, as an improved formulation, but it could not yet completely mask bitter taste by only cyclodextrin.

### DISCLOSURE

### Technical Problem

The problem sought to be solved by the present invention is to completely mask the bitter taste and paralysis of donepezil, etc., which are the disadvantages of the prior art, and also to provide an oral dissolving film formulation to be convenient for an administration.

### Technical Solution

The present invention provides an oral dissolving film formulation in which the bitter taste and paralysis are completely removed by firstly masking of the taste and paralysis of donepezil or the pharmaceutical acceptable salts thereof with cyclodextrin, and then by secondly masking of it with alginate salt, and its dose is convenient.

### Advantageous Effects

According to the present invention, there is a special advantage to obtain an oral dissolving film formulation in which the bitter taste and paralysis of donepezil or pharmaceutically acceptable salts thereof are masked.

### BEST MODE FOR INVENTION

Donepezil which can be used in the present invention can include a free base of donepezil or a pharmaceutically acceptable acid addition salt thereof (hereinafter, donepezil or pharmaceutically acceptable salts thereof is referred to as ‘donepezil’ in the specification, unless it is specifically described to the contrary), and for example, it can be exemplified as hydrochloride, oxalate, hydrobromide, sulfate, nitrate, phosphate, valerate, fumirate, methan sulphonate, benzene sulphonate, toluene and sulphonate, and among these, hydrochloride is the most preferable.

Meanwhile, according to the research of inventors of the present application, it was found that donepezil is changed in a generation of impurity, paralysis and bitter taste depending on pH, and for example, for donepezil hydrochloride, it has an advantage in the light of that the impurity of it is reduced under pH of about 6.5 or less, but has a disadvantage that paralysis and bitter taste are increased, and when pH is greater than about 6.5, it has a disadvantage that the impurity is increased, but paralysis and bitter taste are reduced.

That is, in the light of the impurity in a design of the formulation, it is advantageous that pH is lower than 6.5, and pH is greater than 6.5 in the light of the bitter taste or paralysis, and such compatible properties to each other mean that the rapid dissolving film formulation comprising donepezil cannot be prepared only by simply controlling pH.

Accordingly, the present inventors researched new approaches which can remove bitter taste and paralysis, with inhibiting impurity, and as the result, surprisingly found that when using double adding technique, i.e., by firstly adding cyclodextrin or derivatives thereof, and secondly adding alginic acid or derivatives thereof, the impurity is lowered, and bitter taste and paralysis are also completely removed.

Therefore, the first technical feature of the present invention it characterized in a rapid dissolving film formulation for treating dementia, which comprises cyclodextrin or derivatives thereof, alginic acid or the salts thereof and a substrate for forming films, as the rapid dissolving film comprising donepezil or pharmaceutically acceptable salts thereof.

Specifically, in the present invention, it contains cyclodextrin or the derivatives thereof in order to mask the bitter taste and paralysis of donepezil, and in this case, it can contain α-, β-, or γ-type cyclodextrin, and preferably it can use β-cyclodextrin. The β-cyclodextrin may include hydroxypropylbetadex.

Cyclodextrin can inhibit a generation of impurity, and paralysis and bitter taste, but conversely, when considering the overall physical property of the film formulation, it was found that when it is used in an excess amount, a viscosity is increased (stickiness) and thus productivity is sharply decreased. That is, when it was used as a range which can inhibit the generation of impurity, the effect for inhibiting the impurity, and the reduction of paralysis and bitter taste could be achieved, but it was difficult to prepare the film formulation which is eventually the final formulation, and when cyclodextrin was used in the amount of the range to prepare the film formulation, the effect for inhibiting paralysis and bitter taste was not sufficient.

But, as a result of the persistent research of the present inventors, when cyclodextrin is simultaneously used with alginic acid or the salts thereof, it has been surprisingly found that impurity can be inhibited, and also, paralysis and bitter taste can be completely masked, without affecting the productivity. The alginic acid or the salts thereof which can be used in the present invention can be selected from for example, sodium alginate, or calcium alginate.

In particular, when cyclodextrin is used simultaneously with alginic acid or the salts thereof, there is a characteristic that paralysis is gradually decreased over about 4 weeks after preparing the final formulation. That is, when considering that there is reached to the level that paralysis and bitter taste are not experienced even in the same sample after 4 weeks rather than soon after its preparation, it can be noticed that a simultaneous use of cyclodextrin and alginic acid or the salts thereof can maximize the paralysis and mask effect of bitter taste, by a light change, and such effect has not been known in the prior art.

That is, when taking all the above matters, it is impossible to reduce impurity and mask the bitter taste and paralysis by donepezil with cyclodextrin, in relative to film formulation, but the present invention is differentiated from the technique of the prior art in view of that the final physical property was excellent and also the impurity could be inhibited and bitter taste and paralysis can be completely masked by using cyclodextrin and alginate simultaneously.

In addition, it is estimated that such an interaction between cyclodextrin and alginate is due to the firstly adding of cyclodextrin and then secondly adding of alginate.

In the present invention, a combination amounts of cyclodextrin and alginate can be used as about 1:0.5˜1:8 and 1:05˜1:4, in a percent by weight (% w/w) for effective ingredients, respectively.

Furthermore, in order to lower the impurity, and mask bitter taste and paralysis, when cyclodextrin and alginate are used simultaneously, and also ingredients such as crospovidone, sodium chloride, dibutylhydroxytoluene, etc. are used in alone or in combination, the more excellent effect can be achieved.

The combination ration of crospovidone, sodium hydrochloride and dibuthylhydroxytoluene can be in the range of 1:05˜1:4, 1:1˜1:6 and 1:0.0036˜1:0.125 as a percent by weight (% w/w) in relative to the effective ingredients, respectively.

Accordingly, the present invention relates to an oral dissolving film formulation for treating dementia, which comprises donepezil or the acid addition salt thereof, and cyclodextrin or derivatives thereof, alginic acid or salts thereof and a substrate for forming films, and further relates to a method for preparing the oral dissolving film formulation for dementia, which comprises firstly adding a substrate for forming films to a liquid solution manufactured by firstly adding donepezil or acid addition salt thereof and secondly adding alginic acid or salts thereof to a solution in which cyclodextrin or derivatives thereof is dissolved in a purified water to mask bitter taste and paralysis of donepezil.

The above film formulation can additionally comprise excipient, suspending agent, disintegrating agent, coloring agent, sweetening agent, surfactant, plasticizer, flavoring agent, lubricant, stabilizer and solvent.

Sodium chloride is preferable as the suspending agent, and it is preferable to use crospovidone and titanium oxide by alone or mixing. The disintegrating agent can be used up to about 1% w/w % in relative to the total film formulation, and when titanium oxide is used as the disintegrating agent, it can be used in a ratio of 1:1˜1:5 of titanium oxide:lubricant.

As the coloring agent, titanium oxide, iron oxide sulfate or a pigment recommended by FD&C can be included in an amount of less than about 1% w/w in relative to the total of film formulation.

Sweetening agent is one which can be melted or dissolved in a mouth, and can include one or more selected from sucralose, sucrose, dextrose, fructose, glucose, liquid glucose or maltose.

Surfactant includes polysorbate 20 and polysorbate 80, and among these, polysorbate 20 is preferable to obtain the stable formulation.

Plasticizers are for improving flexibility and brittleness of strip, and selected from glycerin (glycerol), the concentrated glycerin, sorbitol, propylene glycol, a low molecular weight PEG, phthalate polymer such as dimethyl-, diethyl-, dibuthylphthalate, citrate derivative such as tributhyl-, triethyl- or acethylcitrate, triacetin or castor oil, and can be included in the range of about 0-20% w/w in relative to the total of film formulation.

When considering the film cracking, spriting and filling, and the absorption of the drug, glycerin and liquid of sorbitol can be used by mixing in the present invention.

As the excipient (film base), hypromelose, hydroxypropylcellulose, starch or the controlled starch, pullulan, pectin, gelatin, carboxymethylcellulose, and the like can be included, and pullulan is used for stable forming of strip in the present invention.

Flavoring agent can be used by alone or mixing, and menthol oil (I-menthol), cinnamon oil, spearmint oil, vanilla and cocoa oil, coffee and chocolate can be included. It can be used to mask the taste, and can be used up to about 10% w/w in relative to the total of film formulation.

As the antioxidant, dibuthylhydroxytoluene can be included up to about 1% w/w in relative to the total of film formulation in order for the stabilization of the formulation, and as other solvents, the purified water and/or ethanol can be included.

As a saliva stimulant agent, citric acid, malic acid, lactic acid, ascorbic acid or tartaric acid which can increase a generation of saliva to dissolve the formed strip rapidly can be included.

As the lubricant, it can be selected from magnesium stearate, talc, colloidal silicon dioxide, magnesium silicate.

In order to help an understanding of the present invention, Examples are described. The Comparative Examples and Examples are given to easily understand the present invention, but the scope of the present invention is not limited to them.

### Comparative Examples 1-3

### Comparative Example 1

After comparing and reviewing main excipients to set a basic prescription of donepezil chloride oral dissolving film, the results are described in the below table 1.

TABLE 1

| The object | | | | | | | | | | | | | | | | | | | |
|---|---|---|---|---|---|---|---|---|---|---|---|---|---|---|---|---|---|---|---|
| of the | | | | | | | | | | | | | | | | | | | |
| combination | Ingredient | #1 | #2 | #3 | #4 | #5 | #6 | #7 | #8 | #9 | #10 | #11 | #12 | #13 | #14 | #15 | #16 | #17 | #18 |
| Main | Donepezil | 15.00 | 15.00 | 15.00 | 15.00 | 15.00 | 15.00 | 20.00 | 20.00 | 20.00 | 20.00 | 20.00 | 20.00 | 15.00 | 15.00 | 15.00 | 15.00 | 15.00 | 15.00 |
| component | hydrochloride | | | | | | | | | | | | | | | | | | |
| Film- | Fluran | 5.00 | 5.00 | 5.00 | 5.00 | 5.00 | 5.00 | 25.00 | 25.00 | 25.00 | 25.00 | 25.00 | 25.00 | 45.00 | 45.00 | 45.00 | 45.00 | 45.00 | 45.00 |
| forming | | | | | | | | | | | | | | | | | | | |
| agent | | | | | | | | | | | | | | | | | | | |
| Excipient | AvicelPH101 | 45.00 | 0.00 | 0.00 | 0.00 | 0.00 | 0.00 | 20.00 | 0.00 | 0.00 | 0.00 | 0.00 | 0.00 | 5.00 | 0.00 | 0.00 | 0.00 | 0.00 | 0.00 |
| | AvicelPH102 | 0.00 | 45.00 | 0.00 | 0.00 | 0.00 | 0.00 | 0.00 | 20.00 | 0.00 | 0.00 | 0.00 | 0.00 | 0.00 | 5.00 | 0.00 | 0.00 | 0.00 | 0.00 |
| | Lactose | 0.00 | 0.00 | 45.00 | 0.00 | 0.00 | 0.00 | 0.00 | 0.00 | 20.00 | 0.00 | 0.00 | 0.00 | 0.00 | 0.00 | 5.00 | 0.00 | 0.00 | 0.00 |
| | unhydride(#200) | | | | | | | | | | | | | | | | | | |
| | Hydroxy- | 0.00 | 0.00 | 0.00 | 45.00 | 0.00 | 0.00 | 0.00 | 0.00 | 0.00 | 20.00 | 0.00 | 0.00 | 0.00 | 0.00 | 0.00 | 5.00 | 0.00 | 0.00 |
| | prophybetadex | | | | | | | | | | | | | | | | | | |
| | Sodium | 0.00 | 0.00 | 0.00 | 0.00 | 45.00 | 0.00 | 0.00 | 0.00 | 0.00 | 0.00 | 20.00 | 0.00 | 0.00 | 0.00 | 0.00 | 0.00 | 5.00 | 0.00 |
| | alginate | | | | | | | | | | | | | | | | | | |
| | Sodium | 0.00 | 0.00 | 0.00 | 0.00 | 0.00 | 45.00 | 0.00 | 0.00 | 0.00 | 0.00 | 0.00 | 20.00 | 0.00 | 0.00 | 0.00 | 0.00 | 0.00 | 5.00 |
| | hydrochloride | | | | | | | | | | | | | | | | | | |
| Disintegrating | crospovidone | 10.00 | 10.00 | 10.00 | 10.00 | 10.00 | 10.00 | 10.00 | 10.00 | 10.00 | 10.00 | 10.00 | 10.00 | 10.00 | 10.00 | 10.00 | 10.00 | 10.00 | 10.00 |
| agent | | | | | | | | | | | | | | | | | | | |
| Plasticizer | Glycerine/ | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| | Sorbitol liquid | | | | | | | | | | | | | | | | | | |
| Surfactant | Polysorbate20/80 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| Sweetening | Sucralose | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| agent | Aspartame | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| Fragrance | l-Menthol | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 |
| ingredients | Lemon flavor | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 |
| Total mass (%) | | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 |
| Physical | Phase separation | 0 | 0 | 0 | X | X | X | 0 | 0 | 0 | X | X | X | 0 | 0 | 0 | X | X | X |
| property of | Description | Precip- | Precip- | Precip- | Good | Good | Good | Precip- | Precip- | Precip- | Good | Good | Good | Good | Good | Mild | Good | Good | Good |
| crude liquid | | itation | itation | itation | | | | itation | itation | itation | | | | | | precip- | | | |
| | | | | | | | | | | | | | | | | itation | | | |
| Physical | Statue of | X | X | X | 0 | 0 | 0 | X | X | X | 0 | 0 | 0 | 0 | 0 | 0 | 0 | 0 | 0 |
| property | the formation | | | | | | | | | | | | | | | | | | |
| of film | of film | | | | | | | | | | | | | | | | | | |
| | FOLDING TEST | — | — | — | 5 | 5 | 4 | — | — | — | 5 | 5 | 5 | 1 | 1 | 1 | 5 | 5 | 5 |

Folding Test—represented as a numerical value regarding the tendency that the film is cracked or broken, when the film is folded in 180 degree.

1=The film is cracked or broken, when it is folded once.

2=The film is cracked or broken, when it is folded twice.

3=The film is cracked or broken, when it is folded three times.

4=The film is cracked or broken, when it is folded four times.

5=The film is cracked or broken, when it is folded five or more times.

*Phase separation—The phenomenon in which the solid and liquid phases are not homogeneously mixed, and are separated.

*Precipitation—The phenomenon in which the solid is settled on the bottom of the container.

0 The given item is occurred. X The given item is not occurred.

### A Result of the Comparative Example

From the above Comparative Example, it can be seen that 5-45 wt. % of pullulan, 5-45 wt. % of hydroxypropylbetadex, 5-45 wt. % of sodium alginate and 5-45 wt. % of sodium chloride can be included as the main excipients.

The chemical properties of donepezil hydrochloride are as follows.

TABLE 2

| | | Occurrence | | | |
|---|---|---|---|---|---|
| | | of the | | | |
| | | impurity | Paralysis | Bitter taste | Productivity |
| pH | About 6.5 | Reduction | Increase | Increase | Middle |
| | or less | | | | |
| | About 6.5 | Increase | Reduction | Reduction | Decrease |
| | or more | | | | (occurrence of |
| | | | | | mass in the |
| | | | | | crude liquid, A |
| | | | | | stick of taffy- |
| | | | | | phenomenon |

Accordingly, it is needed for the development of the product that the bioequivalence (Cmax, AUC) is maintained like the existing formulation, the occurrence of the impurity is inhibited, the dose compliance of the patient in relation to paralysis and bitter taste is excellent, and mass production is possible.

The characteristic feature of the main excipients of the present invention—In order to achieve the object of the above formulation research, it can be seen that the main excipients of the below Table 3 are essential elements.

TABLE 3

| | | | | Level of difficulty | |
|---|---|---|---|---|---|
| | Occurrence | | | for the process | |
| | of the | | | of the crude | |
| | impurity | Paralysis | Bitter taste | liquid | Productivity |
| Hydroxypropylbetadex | Inhibition | Inhibition, | Inhibition | Good | Sharply decrease, |
| | (Using in | Strong | | | sticky, Unable to |
| | excess) | | | | produce when it is |
| | | | | | present in excess |
| Sodium | . | Inhibition, | . | Increase | Decrease, Excess |
| alginate | | middle | | (increase of the | of the purified |
| | | | | purified water, | water, Breaking of |
| | | | | increase of | the film, Deviation |
| | | | | viscosity -> | of the mass, |
| | | | | decrease of the | Unable to produce |
| | | | | drying efficiency | when it is present |
| | | | | | in excess, |
| Crospovidon | . | Inhibition, | Inhibition, | Good | Decrease, Uneven |
| | | middle | low | | of the film |
| | | | | | description |
| Sodium | . | . | Inhibition | Good | , |
| chloride | | | | | |
| Dibuthylhydroxytoluene | Inhibition | — | — | — | — |

That is, when five main excipients are properly combined, the paralysis and productivity can be secured.

Characteristic in the main process of the present invention—In order to achieve the object of the above formulation research, it can be seen that the following processes are especially needed.

1) Addition of the main ingredients—In order to mask the bitter taste and paralysis, there are processes which comprise firstly adding hydroxypropylbetadex, and secondly adding sodium alginate.

2) Use of the purified water of the room temperature—In order to inhibit the occurrence of the impurity, the purified water having the temperature of 50° C. or less or the room temperature is used.

### Comparative Example 2

Comparative Example to obtain the best ratio by the combination of the main excipients of the present invention was performed, and the results are illustrated in the below Tables 4 and 5.

TABLE 4

| The object | | | | | | | | | | |
|---|---|---|---|---|---|---|---|---|---|---|
| of the | | | | | | | | | | |
| combination | Ingredients | CONTROL | #16 | #10 | #4 | #17 | #11 | #5 | #18 | #12 |
| Main | Donepezil | 40.00 | 15.00 | 20.00 | 15.00 | 15.00 | 20.00 | 15.00 | 15.00 | 20.00 |
| ingredients | hydrochloride | | | | | | | | | |
| Film- | Pullulan | 25.00 | 45.00 | 25.00 | 5.00 | 45.00 | 25.00 | 5.00 | 45.00 | 25.00 |
| forming | | | | | | | | | | |
| agent | | | | | | | | | | |
| Excipients | Hydroxy- | 0.00 | 5.00 | 20.00 | 45.00 | 0.00 | 0.00 | 0.00 | 0.00 | 0.00 |
| | propylbetadex | | | | | | | | | |
| | Sodium alginate | 0.00 | 0.00 | 0.00 | 0.00 | 5.00 | 20.00 | 45.00 | 0.00 | 0.00 |
| | Sodium alginate | 0.00 | 0.00 | 0.00 | 0.00 | 5.00 | 20.00 | 45.00 | 0.00 | 0.00 |
| | Sodium chloride | 0.00 | 0.00 | 0.00 | 0.00 | 0.00 | 0.00 | 0.00 | 5.00 | 20.00 |
| Disingredients | Crospovidone | 10.00 | 10.00 | 10.00 | 10.00 | 10.00 | 10.00 | 10.00 | 10.00 | 10.00 |
| Plasticizer | Glycerin/ | 5.00 | 5.00 | 5.00 | 5.00 | 5.00 | 5.00 | 5.00 | 5.00 | 5.00 |
| | Sorbitol liquid | | | | | | | | | |
| Surfactant | Polysorbate 20/80 | 5.00 | 5.00 | 5.00 | 5.00 | 5.00 | 5.00 | 5.00 | 5.00 | 5.00 |
| Sweetening | Sucralose | 5.00 | 5.00 | 5.00 | 5.00 | 5.00 | 5.00 | 5.00 | 5.00 | 5.00 |
| agent | Aspartame | 5.00 | 5.00 | 5.00 | 5.00 | 5.00 | 5.00 | 5.00 | 5.00 | 5.00 |
| Fragrance | l-menthol | 2.00 | 2.00 | 2.00 | 2.00 | 2.00 | 2.00 | 2.00 | 2.00 | 2.00 |
| ingredients | Lemon flavor | 3.00 | 3.00 | 3.00 | 3.00 | 3.00 | 3.00 | 3.00 | 3.00 | 3.00 |
| Total mass (%) | | 100.00 | 100.00 | 100.00 | 100.00 | 100.00 | 100.00 | 100.00 | 100.00 | 100.00 |
| Physical | Phase separation | X | X | X | X | X | X | X | X | X |
| properties | Description | Good | Good | Good | Good | Good | Good | Good | Good | Good |
| of the | | | | | | | | | | |
| crude liquid | | | | | | | | | | |
| Physical | Status of forming | 0 | 0 | 0 | 0 | 0 | 0 | 0 | 0 | 0 |
| property | the film | | | | | | | | | |
| of the film | FOLDING TEST | 5 | 5 | 5 | 5 | 5 | 5 | 5 | 5 | 5 |
| Sensory | Bitter taste | 1.1 | 2.1 | 2.2 | 3.1 | 2.1 | 2.2 | 3.4 | 2.2 | 3.1 |
| test | and paralysis | | | | | | | | | |
| | Mouthfeel | 4.1 | 4.2 | 4.1 | 4.1 | 4.1 | 4.2 | 4.1 | 4.2 | 4.2 |
| | (feeling | | | | | | | | | |
| | of irritation/ | | | | | | | | | |
| | after sensation) | | | | | | | | | |
| | Side-effect | 1.2 | 2.1 | 3.2 | 4.8 | 2.3 | 4.2 | 4.9 | 2.1 | 3.1 |
| | (headache/ | | | | | | | | | |
| | nausea | | | | | | | | | |
| Production | Stickiness | X | X | X | Δ | X | X | X | X | X |
| trouble | Heterogeneous | X | X | X | X | X | X | Δ | X | X |
| | dryness | | | | | | | | | |

TABLE 5

| Object | | | | | | | | | | | | | |
|---|---|---|---|---|---|---|---|---|---|---|---|---|---|
| of the | | | | | | | | | | | | | |
| combination | Ingredients | CONTROL | #19 | #6 | #20 | #21 | #22 | #23 | #24 | #25 | #26 | #27 | #28 |
| Main | Donepezil | 40.0 | 15.0 | 15.0 | 10.0 | 10.0 | 10.0 | 10.0 | 10.0 | 10.0 | 10.0 | 10.0 | 10.0 |
| ingredients | hydrochloride | | | | | | | | | | | | |
| Film | Pullulan | 25.00 | 20.00 | 5.00 | 15.00 | 15.00 | 15.00 | 15.00 | 15.00 | 15.00 | 5.00 | 5.00 | 5.00 |
| forming | | | | | | | | | | | | | |
| agent | | | | | | | | | | | | | |
| Excipient | Hydroxy- | 0.00 | 0.00 | 0.00 | 5.00 | 5.00 | 20.00 | 15.00 | 30.00 | 30.00 | 20.00 | 20.00 | 20.00 |
| | propylbetadex | | | | | | | | | | | | |
| | Sodium | 0.00 | 0.00 | 0.00 | 30.00 | 30.00 | 15.00 | 20.00 | 5.00 | 5.00 | 15.00 | 15.00 | 15.00 |
| | alginate | | | | | | | | | | | | |
| | Sodium | 0.00 | 30.00 | 45.00 | 5.00 | 5.00 | 5.00 | 5.00 | 5.00 | 5.00 | 5.00 | 15.00 | 20.00 |
| | chloride | | | | | | | | | | | | |
| Disintegrat- | Crospovidon | 10.00 | 10.00 | 10.00 | 10.00 | 10.00 | 10.00 | 10.00 | 10.00 | 10.00 | 20.00 | 10.00 | 5.00 |
| ing agent | | | | | | | | | | | | | |
| Plasticizer | Glycerine/ | 5.00 | 5.00 | 5.00 | 5.00 | 5.00 | 5.00 | 5.00 | 5.00 | 5.00 | 5.00 | 5.00 | 5.00 |
| | Sorbitol liquid | | | | | | | | | | | | |
| Surfactant | Polysorbate | 5.00 | 5.00 | 5.00 | 5.00 | 5.00 | 5.00 | 5.00 | 5.00 | 5.00 | 5.00 | 5.00 | 5.00 |
| | 20/80 | | | | | | | | | | | | |
| Sweetening | Sucralose | 5.00 | 5.00 | 5.00 | 5.00 | 5.00 | 5.00 | 5.00 | 5.00 | 5.00 | 5.00 | 5.00 | 5.00 |
| agent | Aspartame | 5.00 | 5.00 | 5.00 | 5.00 | 5.00 | 5.00 | 5.00 | 5.00 | 5.00 | 5.00 | 5.00 | 5.00 |
| Fragrance | l-menthol | 2.00 | 2.00 | 2.00 | 2.00 | 2.00 | 2.00 | 2.00 | 2.00 | 2.00 | 2.00 | 2.00 | 2.00 |
| ingredients | Lemon flavor | 3.00 | 3.00 | 3.00 | 3.00 | 3.00 | 3.00 | 3.00 | 3.00 | 3.00 | 3.00 | 3.00 | 3.00 |
| Total mass (%) | | 100.00 | 100.00 | 100.00 | 100.00 | 100.00 | 100.00 | 100.00 | 100.00 | 100.00 | 100.00 | 100.00 | 100.00 |
| Physical | Phase | X | X | X | X | X | X | X | X | X | X | X | X |
| properties | separation | | | | | | | | | | | | |
| of the | Description | Good | Good | Good | Good | Good | Good | Good | Good | Good | Good | Good | Good |
| crude liquid | | | | | | | | | | | | | |
| Physical | Status of | 0 | 0 | 0 | 0 | 0 | 0 | 0 | 0 | 0 | 0 | 0 | 0 |
| property | forming | | | | | | | | | | | | |
| of the film | the film | | | | | | | | | | | | |
| | FOLDING | 5 | 5 | 4 | 5 | 5 | 5 | 5 | 5 | 5 | 5 | 5 | 5 |
| | TEST | | | | | | | | | | | | |
| Sensory | Bitter taste | 1.1 | 3.1 | 3.9 | 4.5 | 4.5 | 4.5 | 4.5 | 4.5 | 4.5 | 4.7 | 4.7 | 4.7 |
| test | and paralysis | | | | | | | | | | | | |
| | Mouthfeel | 4.1 | 4.1 | 3.9 | 4.1 | 4.2 | 3.9 | 4.1 | 4.1 | 4.1 | 4.2 | 4.1 | 4.2 |
| | (feeling of | | | | | | | | | | | | |
| | irritation/ | | | | | | | | | | | | |
| | after sensation) | | | | | | | | | | | | |
| | Side effect | 1.2 | 3.2 | 3.2 | 4.5 | 4.5 | 4.5 | 4.5 | 4.5 | 4.5 | 4.7 | 4.6 | 4.6 |
| | (headache/ | | | | | | | | | | | | |
| | nausea | | | | | | | | | | | | |
| Production | Stickiness | X | X | X | X | X | X | X | X | X | X | X | X |
| trouble | Heterogeneous | X | X | Δ | X | X | X | X | X | X | X | X | X |
| | dryness | | | | | | | | | | | | |

Valuation Criteria

Phase separation: As the same as the description of Table 1

Bitter taste paralysis: 1 (strong paralysis), 2 (paralysis), 3 (little paralysis), 4 (something feeling), 5 (no paralysis)

Mouthfeel: 1 (strong feeling of irritation), 2 (feeling of irritation), 3 (mild feeling of irritation), 4 (something feeling), 5 (no feeling of irritation)

Side effect: 1 (strong headache, nausea), 2 (headache, nausea), 3 (little headache, nausea), 4 (something feeling), 5 (no side effect)

Other: 0 (Occurrence of the given item) X (No occurrence of the given item)

[Results of the Comparative Experiment]

The optimal proportion of the main excipients can be deduced from the above Comparative Experiment, and in particular, it can be seen that the bitter taste and paralysis are completely removed by using both of cyclodextrin and alginate.

The use range of the main excipients is summarized from the above Comparative examples as the following Table 6.

TABLE 6

| No. | Excipients | Item | Range |
|---|---|---|---|
| 1. | Hydroxyprophybetadex | Contrast with API:HP-betadex film | 1:0.5~1:8 4.0~33.33 w/w/% |
| | | weight | |
| 2. | Sodium alginate | Contrast with API:Sodium alginate | 1:0.5~1:4 4.0~16.67 w/w/% |
| | | film weight | |
| 3. | Crospovidone | Contrast with API:crospovidone | 1:0.5~1:4 4.0~16.67 w/w/% |
| | | film weight | |
| 4. | Sodium | Contrast with API:Sodium chloride | 1:1~1:6 8.0~25.0 w/w/% |
| | hydrochloride | film weight | |
| 5. | Pullulan | Contrast with API:Pullulan film | 1:1~1:10 8.0~41.67 w/w/% |
| | | weight | |
| 6. | Dibuthylhydroxytoluene | Contrast with API:BHT film weight | 1:0.0036~1:0.125 |
| | | | 0.03~0.5 w/w/% |
| 7. | Plasticizer (Glycerin, | Contrast with API:Plasticizer film | 1:0.3~1:4 2.4~16.67 w/w/% |
| | Sorbitol) | weight | |

1) Hydroxypropylbetadex went through adding process to increase the poor sensuality and stability of the raw material for donepezil or the pharmaceutically acceptable salt thereof,

2) Sodium alginate not only improved the status of the film, simultaneously with increasing the sensuality, but also increased the sensuality and productivity by being mixed with hydroxypropylbetadex in the optimal ratio.

3) In addition, crospovidone increases not only the disintegration of the film but also the sensuality.

4) Sodium chloride enhanced the sensuality.

| The object | | | | | | | | | | | | | | | | | | |
|---|---|---|---|---|---|---|---|---|---|---|---|---|---|---|---|---|---|---|
| of the | | | | | | | | | | | | | | | | | | |
| combination | Ingredients | CONTROL(#26) | #29 | #30 | #31 | #32 | #33 | #34 | #35 | #36 | #37 | #38 | #39 | #40 | #41 | #42 | #43 | #44 |
| Main | Donepezil | 10.00 | 9.50 | 9.00 | 8.50 | 8.00 | 9.40 | 9.40 | 9.20 | 9.00 | 8.90 | 8.40 | 7.45 | 6.45 | 8.95 | 7.95 | 6.45 | 4.45 |
| ingredient | hydrochloride | | | | | | | | | | | | | | | | | |
| Film - | Pullulan | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| forming | | | | | | | | | | | | | | | | | | |
| agent | | | | | | | | | | | | | | | | | | |
| Excipients | Hydroxy- | 20.00 | 20.00 | 20.00 | 20.00 | 20.00 | 20.00 | 20.00 | 20.00 | 20.00 | 20.00 | 20.00 | 20.00 | 20.00 | 20.00 | 20.00 | 20.00 | 20.00 |
| | prophybetadex | | | | | | | | | | | | | | | | | |
| | Sodium alginate | 15.00 | 15.00 | 15.00 | 15.00 | 15.00 | 15.00 | 15.00 | 15.00 | 15.00 | 15.00 | 15.00 | 15.00 | 15.00 | 15.00 | 15.00 | 15.00 | 15.00 |
| | Sodium | 5.00 | 5.00 | 15.00 | 20.00 | 20.00 | 20.00 | 20.00 | 20.00 | 20.00 | 20.00 | 20.00 | 20.00 | 20.00 | 20.00 | 20.00 | 20.00 | 20.00 |
| | hydrochloride | | | | | | | | | | | | | | | | | |
| Disintegrating | Crospovidone | 20.00 | 20.00 | 10.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| agent | Titanium oxide | 0.00 | 0.50 | 1.00 | 1.50 | 2.00 | 0.50 | 0.50 | 0.50 | 0.50 | 0.50 | 0.50 | 0.50 | 0.50 | 0.50 | 0.50 | 0.50 | 0.50 |
| Plasticizer | Glycerin/ | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| | Sorbitol liquid | | | | | | | | | | | | | | | | | |
| Surfactant | Polysorbate 20/80 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| Sweetening | Sucralose | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| agent | Aspartame | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| Fragrance | l-menthol | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 |
| ingredients | | | | | | | | | | | | | | | | | | |
| | Lemon flavor | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 |
| | (strawberry | | | | | | | | | | | | | | | | | |
| | flavor, | | | | | | | | | | | | | | | | | |
| | mint flavor) | | | | | | | | | | | | | | | | | |
| Antioxidant | BHT | 0.00 | 0.00 | 0.00 | 0.00 | 0.00 | 0.03 | 0.10 | 0.25 | 0.50 | 0.05 | 0.05 | 0.05 | 0.05 | 0.05 | 0.05 | 0.05 | 0.05 |
| Lubricants | Magnesium | 0.00 | 0.00 | 0.00 | 0.00 | 0.00 | 0.00 | 0.00 | 0.00 | 0.00 | 0.50 | 1.00 | 2.00 | 3.00 | 0.00 | 0.00 | 0.00 | 0.00 |
| | stearate | | | | | | | | | | | | | | | | | |
| | Talc | 0.00 | 0.00 | 0.00 | 0.00 | 0.00 | 0.00 | 0.00 | 0.00 | 0.00 | 0.50 | 1.00 | 2.00 | 3.00 | 0.00 | 0.00 | 0.00 | 0.00 |
| Total mass (%) | | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 |
| Physical | Phase separation | X | X | X | X | X | X | X | X | X | X | X | X | 0 | X | X | X | 0 |
| property | Statue of | Good | Good | Good | Good | Good | Good | Good | Good | Good | Good | Good | Good | Good | Bad | Good | Good | Sleep |
| of the | the phase | | | | | | | | | | | | | | | | | |
| crude liquid | | | | | | | | | | | | | | | | | | |
| Physical | Film- forming | 0 | 0 | 0 | 0 | 0 | 0 | 0 | 0 | 0 | 0 | 0 | 0 | — | 0 | 0 | 0 | — |
| property | statues | | | | | | | | | | | | | | | | | |
| of the film | FOLDING TEST | 5 | 5 | 5 | 5 | 5 | 5 | 5 | 5 | 5 | 5 | 5 | 5 | — | 5 | 5 | 5 | — |
| | Disintegration | Within | Within | Within | Within | Within | Within | Within | Within | Within | Within | Within | Within | Within | Within | Within | Within | Within |
| | time | 1.5 min | 1 min | 50 sec | 50 sec | 40 sec | 1 min | 1 min | 1 min | 1 min | 1 min | 1 min | 1 min | 1 min | 1 min | 1 min | 1 min | 1 min |
| | Total impurity | 0.28 | 0.30 | 0.31 | 0.38 | 0.64 | 0.27 | 0.29 | 0.24 | 0.28 | 0.30 | 0.25 | 0.27 | — | 0.26 | 0.28 | 0.24 | — |
| | (preservation | | | | | | | | | | | | | | | | | |
| | at 80° C./ | | | | | | | | | | | | | | | | | |
| | for 9 days), | | | | | | | | | | | | | | | | | |
| | Criterion 1, | | | | | | | | | | | | | | | | | |
| | 1.0% or less | | | | | | | | | | | | | | | | | |
| Sensual test | Mouth feel | 4 | — | — | 4 | — | — | — | 4 | — | — | — | — | — | — | — | — | — |
| | (feeling of | | | | | | | | | | | | | | | | | |
| | irritation/ | | | | | | | | | | | | | | | | | |
| | after sensation) | | | | | | | | | | | | | | | | | |
| | Side effect | 4.7 | — | — | 4.7 | — | — | — | 4.7 | — | — | — | — | — | — | — | — | — |
| | (headache/ | | | | | | | | | | | | | | | | | |
| | nausea) | | | | | | | | | | | | | | | | | |
| Production | Stickiness | X | X | X | X | — | X | X | X | X | X | X | X | — | X | X | X | — |
| trouble | Homogeneous | X | X | X | X | — | X | X | X | X | X | X | X | — | X | X | X | — |
| | dryness | | | | | | | | | | | | | | | | | |
| | Detachment | 0 | 0 | 0 | 0 | — | 0 | 0 | 0 | 0 | 0 | 0 | 0 | — | 0 | 0 | 0 | — |

5) A specific gravity of pullulan played a great role in forming the film, and the specific gravity optimized for the productivity could be found.

6) In the case of dibuthyhydroxytoluen, there are lots of antioxidants, but the most effective combination of it with antioxidant could be found.

7) When the plasticizer was added in a few amounts, the film was broken and when it was added in a lot, it became sticky and the productivity is low, and thus, the productivity could be enhanced by finding the optimal ratio of it.

### Comparative Example 3

Furthermore, the Comparative Example was performed in order to deduce the optimal combination ratio of titanium oxide, antioxidant and lubricant, which are the combination ingredients of main excipients of the present invention, and the results are described in Table 7.

TABLE 7

| The object | | | | | | | | | | | | | | | | | | |
|---|---|---|---|---|---|---|---|---|---|---|---|---|---|---|---|---|---|---|
| of the | | | | | | | | | | | | | | | | | | |
| combination | Ingredients | CONTROL(#26) | #29 | #30 | #31 | #32 | #33 | #34 | #35 | #36 | #37 | #38 | #39 | #40 | #41 | #42 | #43 | #44 |
| Main | Donepezil | 10.00 | 9.50 | 9.00 | 8.50 | 8.00 | 9.40 | 9.40 | 9.20 | 9.00 | 8.90 | 8.40 | 7.45 | 6.45 | 8.95 | 7.95 | 6.45 | 4.45 |
| ingredient | hydrochloride | | | | | | | | | | | | | | | | | |
| Film - | Pullulan | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| forming | | | | | | | | | | | | | | | | | | |
| agent | | | | | | | | | | | | | | | | | | |
| Excipients | Hydroxy- | 20.00 | 20.00 | 20.00 | 20.00 | 20.00 | 20.00 | 20.00 | 20.00 | 20.00 | 20.00 | 20.00 | 20.00 | 20.00 | 20.00 | 20.00 | 20.00 | 20.00 |
| | prophybetadex | | | | | | | | | | | | | | | | | |
| | Sodium alginate | 15.00 | 15.00 | 15.00 | 15.00 | 15.00 | 15.00 | 15.00 | 15.00 | 15.00 | 15.00 | 15.00 | 15.00 | 15.00 | 15.00 | 15.00 | 15.00 | 15.00 |
| | Sodium | 5.00 | 5.00 | 15.00 | 20.00 | 20.00 | 20.00 | 20.00 | 20.00 | 20.00 | 20.00 | 20.00 | 20.00 | 20.00 | 20.00 | 20.00 | 20.00 | 20.00 |
| | hydrochloride | | | | | | | | | | | | | | | | | |
| Disintegrating | Crospovidone | 20.00 | 20.00 | 10.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| agent | Titanium oxide | 0.00 | 0.50 | 1.00 | 1.50 | 2.00 | 0.50 | 0.50 | 0.50 | 0.50 | 0.50 | 0.50 | 0.50 | 0.50 | 0.50 | 0.50 | 0.50 | 0.50 |
| Plasticizer | Glycerin/ | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| | Sorbitol liquid | | | | | | | | | | | | | | | | | |
| Surfactant | Polysorbate 20/80 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| Sweetening | Sucralose | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| agent | Aspartame | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 | 5.00 |
| Fragrance | l-menthol | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 | 2.00 |
| ingredients | | | | | | | | | | | | | | | | | | |
| | Lemon flavor | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 | 3.00 |
| | (strawberry | | | | | | | | | | | | | | | | | |
| | flavor, | | | | | | | | | | | | | | | | | |
| | mint flavor) | | | | | | | | | | | | | | | | | |
| Antioxidant | BHT | 0.00 | 0.00 | 0.00 | 0.00 | 0.00 | 0.03 | 0.10 | 0.25 | 0.50 | 0.05 | 0.05 | 0.05 | 0.05 | 0.05 | 0.05 | 0.05 | 0.05 |
| Lubricants | Magnesium | 0.00 | 0.00 | 0.00 | 0.00 | 0.00 | 0.00 | 0.00 | 0.00 | 0.00 | 0.50 | 1.00 | 2.00 | 3.00 | 0.00 | 0.00 | 0.00 | 0.00 |
| | stearate | | | | | | | | | | | | | | | | | |
| | Talc | 0.00 | 0.00 | 0.00 | 0.00 | 0.00 | 0.00 | 0.00 | 0.00 | 0.00 | 0.50 | 1.00 | 2.00 | 3.00 | 0.00 | 0.00 | 0.00 | 0.00 |
| Total mass (%) | | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 |
| Physical | Phase separation | X | X | X | X | X | X | X | X | X | X | X | X | 0 | X | X | X | 0 |
| property | Statue of | Good | Good | Good | Good | Good | Good | Good | Good | Good | Good | Good | Good | Good | Bad | Good | Good | Sleep |
| of the | the phase | | | | | | | | | | | | | | | | | |
| crude liquid | | | | | | | | | | | | | | | | | | |
| Physical | Film- forming | 0 | 0 | 0 | 0 | 0 | 0 | 0 | 0 | 0 | 0 | 0 | 0 | — | 0 | 0 | 0 | — |
| property | statues | | | | | | | | | | | | | | | | | |
| of the film | FOLDING TEST | 5 | 5 | 5 | 5 | 5 | 5 | 5 | 5 | 5 | 5 | 5 | 5 | — | 5 | 5 | 5 | — |
| | Disintegration | Within | Within | Within | Within | Within | Within | Within | Within | Within | Within | Within | Within | Within | Within | Within | Within | Within |
| | time | 1.5 min | 1 min | 50 sec | 50 sec | 40 sec | 1 min | 1 min | 1 min | 1 min | 1 min | 1 min | 1 min | 1 min | 1 min | 1 min | 1 min | 1 min |
| | Total impurity | 0.28 | 0.30 | 0.31 | 0.38 | 0.64 | 0.27 | 0.29 | 0.24 | 0.28 | 0.30 | 0.25 | 0.27 | — | 0.26 | 0.28 | 0.24 | — |
| | (preservation | | | | | | | | | | | | | | | | | |
| | at 80° C./ | | | | | | | | | | | | | | | | | |
| | for 9 days), | | | | | | | | | | | | | | | | | |
| | Criterion 1, | | | | | | | | | | | | | | | | | |
| | 1.0% or less | | | | | | | | | | | | | | | | | |
| Sensual test | Mouthfeel | 4 | — | — | 4 | — | — | — | 4 | — | — | — | — | — | — | — | — | — |
| | (feeling of | | | | | | | | | | | | | | | | | |
| | irritation/ | | | | | | | | | | | | | | | | | |
| | after sensation) | | | | | | | | | | | | | | | | | |
| | Side effect | 4.7 | — | — | 4.7 | — | — | — | 4.7 | — | — | — | — | — | — | — | — | — |
| | (headache/ | | | | | | | | | | | | | | | | | |
| | nausea) | | | | | | | | | | | | | | | | | |
| Production | Stickiness | X | X | X | X | — | X | X | X | X | X | X | X | — | X | X | X | — |
| trouble | Homogeneous | X | X | X | X | — | X | X | X | X | X | X | X | — | X | X | X | — |
| | dryness | | | | | | | | | | | | | | | | | |
| | Detachment | 0 | 0 | 0 | 0 | — | 0 | 0 | 0 | 0 | 0 | 0 | 0 | — | 0 | 0 | 0 | — |

Valuation basis: as the same as the description of Tables 1, 4 and 5

### Results of the Comparative Example

It is preferable that the disintegrating agent, titanium oxide is comprised in an amount of 0.5%˜2.0% in relation to the total weight of the solid content (film).

It is preferable that the antioxidant, buthylhydroxytoluene (BHT) is comprised in an amount of 0.03%˜0.5% in relation to the total weight of the solid content (film).

It is preferable that the lubricant, talc is comprised in an amount of 0.5%˜3.0% and magnesium stearate is comprised in an amount of 0.5%˜2.0% in relation to the total weight.

### Formulation Example 1

Donepezil oral dissolving film was prepared with the composition and contents as shown in the below Table 8.

TABLE 8

| Name of the raw material | 10 mg of the allowable amount |
|---|---|
| Donepezil hydrochloride | 10.00 |
| Hydroxypropylbetadex | 20.00 |
| Sodium hydrochloride | 17.00 |
| Crospovidone | 10.00 |
| Titanium oxide | 0.50 |
| Sodium alginate | 5.00 |
| Sucralose | Optimum dose |
| Polysorbate 20 | Optimum dose |
| Glycerin (PALMERA G995) | Optimum dose |
| Sorbitol liquid | Optimum dose |
| Pullulan | Optimum dose |
| Yellow iron oxide | Optimum dose |
| L-menthol | Optimum dose |
| Dibutylhydroxytoluene | 0.05 |
| Ethanol | Optimum dose |
| Purified water | 200.00 |

(Preparation Method)

According to the above prescription amount,

1) Hydroxypropylbetadex was melted at 50° C. or less or the room temperature, and donepezil hydrochloride was mixed (mixture (1)).

2) Ethanol and dibuthyhydroxytoluene (BHT) were dissolved in a separate SUS container-1 and added to the mixture (1), and then stirred (mixture (2)).

3) Then, sodium alginate was added to the mixture (2), homogeneously stirred and mixed, and sodium chloride, crospovidone and sucralose were added and then homogenized (mixture (3)).

4) Suspension of the yellow iron oxide previously grinned and titanium oxide was added to the mixture (3) in the purified water heated to 50° C. in the separate SUS container-3 (mixture (4)).

5) L-menthol was dissolved in the purified water in the separate SUS container-3 and the solution was added to mixture (4) polysorbate 20 was added to the solution to dissolve, and then the substrate for forming films, pullulan, and the plasticizer, glycerin and sorbitol were added one after another. The homogeneous crude liquid of pH 4.0˜5.7 was obtained.

6) After removing a bubble, coating was performed in a constant thickness according to the administration dose and tailored by cutting in the standard scale size to obtain a complete product.

### Preparation Test Example

After administering the preparation 1 according to the present invention (donepezil hydrochloride oral dissolving film 10 mg) and 10 mg of Ariceptevis tablet to 10 subjects, respectively, paralysis, mouth feeling (feeling of irritation/after sensation), side effect (headache/nausea), disintegrating time, preference, and the like, were compared, and then the test results were described in the below Table 10, together with the valuation basis (Table 9).

TABLE 9

| | Score | | | | |
|---|---|---|---|---|---|
| | 1 | 2 | 3 | 4 | 5 |
| Paralysis | Strong | Paralysis | Some | Something | No |
| | paralysis | | paralysis | feeling | paralysis |
| Mouth | Strong | Some | Mild feeling | Something | No feeling |
| feeling | feeling of | feeling of | of irritation | feeling | of |
| (feeling of | irritation | irritation | | | irritation |
| irritation/ | | | | | |
| after | | | | | |
| sensation) | | | | | |
| Side effect | Severe | Headache, | A little | Something | No side |
| (headache/ | headache, | nausea | headache, | feeling | effect |
| nausea) | nausea | | nausea | | |

TABLE 10

| | | | Mouth feeling | | | | | | | | | | | |
|---|---|---|---|---|---|---|---|---|---|---|---|---|---|---|
| | | | (feeling of irritation/ | | | Side effect | | | Disintegrating | | | | | |
| Paralysis | | | after sensation) | | | (headache/nausea) | | | time (sec) | | | Preference | | |
| | Ari- | Prep. | | Ari- | Prep. | | Ari- | Prep. | | Ari- | Prep. | | Ari- | Prep. |
| | ceptevis | Exam- | | ceptevis | Exam- | | ceptevis | Exam- | | ceptevis | Exam- | | ceptevis | Exam- |
| No. | 10 mg | ple 1 | No. | 10 mg | ple 1 | No. | 10 mg | ple 1 | No. | 10 mg | ple 1 | No. | 10 mg | ple 1 |
| 1 | 3 | 4 | 1 | 2 | 3 | 1 | 3 | 4 | 1 | 20 | 20 | 1 | | 0 |
| 2 | 3 | 5 | 2 | 1 | 4 | 2 | 2 | 4 | 2 | 20 | 20 | 2 | | 0 |
| 3 | 4 | 4 | 3 | 5 | 5 | 3 | 5 | 5 | 3 | 15 | 24 | 3 | 0 | |
| 4 | 3 | 5 | 4 | 2 | 4 | 4 | 5 | 5 | 4 | 23 | 20 | 4 | | 0 |
| 5 | 3 | 4 | 5 | 3 | 5 | 5 | 3 | 4 | 5 | 30 | 40 | 5 | | 0 |
| 6 | 4 | 4 | 6 | 5 | 5 | 6 | 4 | 4 | 6 | 10 | 30 | 6 | | 0 |
| 7 | 4 | 5 | 7 | 4 | 4 | 7 | 5 | 5 | 7 | 10 | 35 | 7 | | 0 |
| 8 | 4 | 5 | 8 | 2 | 2 | 8 | 2 | 5 | 8 | 23 | 30 | 8 | | 0 |
| 9 | 2 | 4 | 9 | 3 | 5 | 9 | 2 | 4 | 9 | 43 | 35 | 9 | | 0 |
| 10 | 3 | 5 | 10 | 2 | 3 | 10 | 3 | 5 | 10 | 25 | 20 | 10 | | 0 |
| Average | 3.30 | 4.50 | Average | 2.90 | 4.00 | Average | 3.40 | 4.50 | Average | 21.90 | 27.40 | Total | Total | Total |
| Standard | 0.67 | 0.53 | Standard | 1.37 | 1.05 | Standard | 1.26 | 0.53 | Standard | 9.78 | 7.59 | 10 | 1 | 9 |
| Deviation | | | Deviation | | | Deviation | | | Deviation | | | | | |

### Reference Example

For the Preparation Example 1, the same experimental was performed on the film formulation immediately after the preparation and the film formulation after 4 weeks, and the results were as shown in the below Table 11.

TABLE 11

| Paralysis | | | Side effect (headache/nausea) | | |
|---|---|---|---|---|---|
| | Immediately | | | Immediately | |
| | after | After 4 | | after | After 4 |
| No. | preparation | weeks | No. | preparation | weeks |
| 1 | 4 | 5 | 1 | 4 | 5 |
| 2 | 5 | 5 | 2 | 4 | 5 |
| 3 | 4 | 5 | 3 | 5 | 5 |
| 4 | 5 | 5 | 4 | 5 | 5 |
| 5 | 4 | 5 | 5 | 4 | 5 |
| 6 | 4 | 5 | 6 | 4 | 5 |
| 7 | 5 | 55 | 7 | 5 | 5 |
| 8 | 5 | 5 | 8 | 5 | 5 |
| 9 | 4 | 5 | 9 | 4 | 5 |
| 10 | 5 | 5 | 10 | 5 | 5 |
| Average | 4.50 | 5.00 | Average | 4.50 | 5.00 |
| Standard | 0.53 | 0.00 | Standard | 0.53 | 0.00 |
| Deviation | | | Deviation | | |

(Point of View)

In the light of the property of the preparation formulation of the present invention in which sodium alginate is added, paralysis is likely reduced over time, and it was identified that paralysis, mouth feeling, side effect, and the like, were improved a lot although they were tested on same subjects.

### INDUSTRIAL APPLICABILITY

According to the present invention, since the oral dissolving film agent in which bitter taste and paralysis of donepezil or the pharmaceutically acceptable salts thereof are masked can be obtained, the present invention has the industrial applicability.

1. An oral dissolving film preparation comprising:
donepezil or a pharmaceutically acceptable salt thereof,
cyclodextrin or a derivative thereof,
alginic acid or a salt thereof, and
a substrate for forming films.

2. The film preparation according to claim 1, wherein the film preparation comprises donepezil or the pharmaceutically acceptable salt thereof selected from hydrochloride, oxalate, hydrobromide, sulfate, nitrate, phosphate, valeric acid, fumarate, methansulphonate, benzensulphonate and toluenesulphonate.

3. The film preparation according to claim 1, wherein the film preparation comprises cyclodextrin or a derivative thereof selected from α-cyclodextrin, β-cyclodextrin and γ-cyclodextrin.

4. The film preparation according to claim 3, wherein the cyclodextrin is β-cyclodextrin, and is comprised in the weight ratio of 1:0.5 to 1:8 in relation to donepezil or a pharmaceutically acceptable salt thereof.

5. The film preparation according to claim 1, wherein the film preparation comprises alginic acid or a salt thereof selected from sodium alginate or calcium alginate.

6. The film preparation according to claim 5, wherein the alginate is sodium alginate, and is comprised in the weight ratio of 1:0.5 to 1:4 in relation to donepezil or a pharmaceutically acceptable salt thereof.

7. The film preparation according to claim 1, wherein the film preparation further comprises one selected from excipient, suspending agent, disintegrating agent, coloring agent, sweetening agent, surfactant, plasticizer, fragrance ingredient, stabilizer, lubricant and solvent.

8. The film preparation according to claim 7, wherein the disintegrating agent is titanium oxide.

9. The film preparation according to claim 8, wherein the content ratio of titanium oxide:lubricant is 1:1 to 1:5.

10. A method for preparing an oral dissolving film preparation for dementia, comprising:
covering bitter taste and paralysis of donepezil by firstly adding donepezil or an acid addition salt thereof and secondly adding alginic acid or a salt thereof to a solution in which cyclodextrin or a derivative thereof is dissolved in the purified water;
adding one or more agents selected from excipient, suspending agent, disintegrating agent, coloring agent, sweetening agent, surfactant, plasticizer, fragrance ingredient, stabilizer and solvent to the solution to solve and homogenize it; and
adding a substrate for forming films to the crude liquid solution obtained from the previous step.

11. A method for treating dementia, comprising:
administering to a subject in need thereof a therapeutically effective amount of the film preparation of claim 1.

12. The method according to claim 11, wherein the cyclodextrin is β-cyclodextrin, and is comprised in the weight ratio of 1:0.5 to 1:8 in relation to donepezil or a pharmaceutically acceptable salt thereof.

13. The method according to claim 11, wherein the alginate is sodium alginate, and is comprised in the weight ratio of 1:0.5 to 1:4 in relation to donepezil or a pharmaceutically acceptable salt thereof.

14. The method according to claim 11, wherein the content ratio of titanium oxide:lubricant is 1:1 to 1:5.
